(19) United States
(12) Patent Application Publication
(10) Pub. No.: US 2022/0031719 A1
(43) Pub. Date: Feb. 3, 2022

(54) ANTIVIRAL THERAPEUTIC DRUG COMBINATIONS

(71) Applicants: THE UNIVERSITY OF TULSA, Tulsa, OK (US); BIOCHARGED VENTURES, LLC, Austin, TX (US)

(72) Inventors: Robert J. SHEAFF, Owasso, OK (US); Ian MITCHELL, Bartlesville, OK (US)

(73) Assignees: THE UNIVERSITY OF TULSA, Tulsa, OK (US); BIOCHARGED VENTURES, LLC, Austin, TX (US)

(21) Appl. No.: 17/443,988

(22) Filed: Jul. 29, 2021

Related U.S. Application Data
(60) Provisional application No. 63/058,165, filed on Jul. 29, 2020.

(51) Int. Cl. A61K 31/706; A61K 31/121; A61K 45/06

(52) U.S. Cl. CPC A61K 31/706; A61K 45/06; A61K 31/121

(57) ABSTRACT

Provided herein are compositions and methods for the treatment or prevention of viral infections via the use of chemical substances administered in a combinatory fashion for the suppression, inhibition, and elimination of viral loads. These compositions and methods use a glycolysis inhibitor (e.g., 2-deoxy-D-glucose (2-DG)) in combination with one or more compounds comprising one or more aromatic rings and at least one alkylamino_{(C≤12)} group or substituted alkylamino_{(C≤12)} group.

### REFERENCE TO RELATED APPLICATIONS

The present application claims the priority benefit of U.S. provisional application No. 63/058,165, filed Jul. 29, 2020, the entire contents of which is incorporated herein by reference.

### BACKGROUND

### 1. Field

The present disclosure relates generally to the fields of medicine and virology. More particularly, it concerns compositions and methods for the treatment or prevention of viral infections via the use of chemical substances administered in a combinatory fashion for the suppression, inhibition, and elimination of viral loads as created by mitochondrial dysregulation prior to or post infection.

### 2. Description of Related Art

Severe Acute Respiratory Syndrome Coronavirus-2 (SARS-CoV-2) is the causative agent for COVID-19, a disease that has killed hundreds of thousands and severely impacted the world economy. While much is known about its structure, genome, and method of infectivity, effective treatments remain elusive. The coronavirus is so named due to the presence of spike-like glycoproteins on its surface, which are necessary for the virus to enter host cells. These bind the angiotensin-converting enzyme (ACE-2) cell membrane receptor, allowing the virus to fuse with the cell membrane and enter as an endosome. SARS-CoV-2 has a positive strand RNA genome, which is released into the host cell by acidification and dissociation of associated capsid proteins. The viral RNA now serves as both mRNA for production of viral proteins and template for synthesis of additional genomic copies. New viral particles are assembled and released via exocytosis. The virus manipulates host cell systems throughout its replicative cycle, which represent possible therapeutic targets.

While pharmaceutical treatments have yet to be identified and approved for COVID-19, the FDA issued emergency use authorization for chloroquine phosphate and the derivative hydroxychloroquine sulfate (recently rescinded) and remdesivir. Chloroquine (CQ) and hydroxychloroquine (HCQ) are well characterized anti-malarial drugs used extensively over the past 60 years. Remdesivir (originally developed to treat Ebola) is a pro-drug that in cells is converted to a nucleotide analog that inhibits replication of the viral RNA genome. Some clinical studies suggest that CQ and HCQ act as preventive and therapeutic agents against COVID-19, while others adamantly dispute these claims. The proposed mechanism(s) of action are also controversial. One hypothesis is based on their anti-malarial activity, which involves CQ accumulation in the Plasmodium food vacuole, where its protonation prevents heme detoxification and causes pathogen death. SARS-CoV-2 requires acidification of endosomes for genome release, and so CQ/HCQ could block this step in a similar fashion. More recent evidence suggests HCQ/CQ can also act as ionophoric agents transporting zinc ions into the infected cell, where they inhibit the viral RNA replicase enzyme. Finally, CQ/HCQ have been shown to dampen excess immune responses associated with autoimmune diseases like lupus. They could play a similar role in treating COVID-19, as a hyperactive immune response is sometimes associated with the disease. Both CQ and HCQ have associated side effects such as inducing heart rhythm problems and interaction with other drugs, which raise further questions about their therapeutic value against COVID-19.

### SUMMARY

Provided herein are compositions and methods for the treatment or prevention of viral infections via the use of chemical substances administered in a combinatory fashion for the suppression, inhibition, and elimination of viral loads. These compositions and methods are more efficacious than single therapies alone, and in specific cases induce a synergistic effect both intracellularly and systemically. The therapies use a glycolysis inhibitor (e.g., 2 Deoxy D-glucose (2-DG)) in combination with one or more compounds having one or more aromatic rings and at least one alkylamino_{(C≤12)} group.

In one embodiment, provided herein are methods of treating a patient having a coronavirus infection or preventing a coronavirus infection in a patient, the methods comprising administering to the patient an effective amount of a first pharmaceutical therapy comprising a glycolysis inhibitor and a second pharmaceutical therapy comprising a compound having one or more aromatic rings and at least one alkylamino_{(C≤12)} group or substituted alkylamino_{(C≤12)} group.

In some aspects, the glycolysis inhibitor is 2-deoxy-D-glucose (2-DG). In some aspects, the first pharmaceutical therapy is administered systemically. In some aspects, the first pharmaceutical therapy is administered orally, by inhalation, by injection, intraarterially, or intravenously. In some aspects, the first pharmaceutical therapy is administered at a dosage of from about 25 mg/kg and to about 150 mg/kg. In some aspects, the first pharmaceutical therapy is administered daily.

In some aspects, the compound is epinephrine, diphenhydramine, diphenhydramine hydrochloride, chloroquine, hydroxychloroquine, 3,4-methylenedioxymethamphetamine, chlorpromazine, phenylephrine, disulfiram, rosiglitazone, camostat, clemastine, dextromethorphan, or nicotine. In some aspects, the compound is in its free base form. In some aspects, the second pharmaceutical therapy is administered systemically. In some aspects, the second pharmaceutical therapy is administered orally, by inhalation, by injection, intraarteriallly, or intravenously.

In some aspects, the first pharmaceutical therapy is administered prior to the administration of the second pharmaceutical therapy. In some aspects, the first pharmaceutical therapy is administered after the administration of the second pharmaceutical therapy. In some aspects, the first pharmaceutical therapy and the second pharmaceutical therapy are administered concurrently. In some aspects, the first pharmaceutical therapy and the second pharmaceutical therapy are administered by distinct routes.

In some aspects, the coronavirus is a betacoronavirus. In some aspects, the coronavirus is a severe acute respiratory syndrome-related (SARS) coronavirus. In some aspects, the coronavirus is SARS-CoV-2. In some aspects, the method is a method of treating or preventing COVID-19 in a patient in need thereof. In some aspects, the method inhibits replication of SARS-CoV-2 in the patient.

In some aspects, the methods further comprise administering to patient a second therapy. In some aspects, the second therapy comprises administering to said patient a therapeutically effective amount of a second drug. In some aspects, the second drug is an anti-platelet drug, an anti-coagulation agent, an anti-viral drug, a corticosteroid, a human type I IFN, or a human type III IFN. In some aspects, the second drug is remdesivir.

In one embodiment, provided herein are pharmaceutical compositions comprising: (a) a glycolysis inhibitor; (b) a compound having one or more aromatic rings and at least one alkylamino_{(C≤12)} group or substituted alkylamino_{(C≤12)} group; and (c) a pharmaceutically acceptable carrier. In some aspects, the glycolysis inhibitor is 2-deoxy-D-glucose (2-DG). In some aspects, the compound having one or more aromatic rings and at least one alkylamino_{(C≤12)} group or substituted alkylamino_{(C≤12)} group is epinephrine, diphenhydramine, diphenhydramine hydrochloride, chloroquine, hydroxychloroquine, 3,4-methylenedioxymethamphetamine, chlorpromazine, phenylephrine, disulfiram, rosiglitazone, camostat, clemastine, dextromethorphan, or nicotine. In some aspects, the compound having one or more aromatic rings and at least one alkylamino_{(C≤12)} group or substituted alkylamino_{(C≤12)} is in its free base form. In some aspects, the pharmaceutical compositions are for use in treating or preventing a coronavirus infection in a patient.

In one embodiment, provided herein are kits comprising: (a) a first pharmaceutical composition comprising a glycolysis inhibitor; (b) a second pharmaceutical composition comprising a compound having one or more aromatic rings and at least one alkylamino_{(C≤12)} group or substituted alkylamino_{(C≤12)} group, wherein the first pharmaceutical composition and the second pharmaceutical composition are provided as separate components of the kit. In some aspects, the glycolysis inhibitor is 2-deoxy-D-glucose (2-DG). In some aspects, the compound having one or more aromatic rings and at least one alkylamino_{(C≤12)} group or substituted alkylamino_{(C≤12)} group is epinephrine, diphenhydramine, diphenhydramine hydrochloride, chloroquine, hydroxychloroquine, 3,4-methylenedioxymethamphetamine, chlorpromazine, phenylephrine, disulfiram, rosiglitazone, camostat, clemastine, dextromethorphan, or nicotine. In some aspects, the compound having one or more aromatic rings and at least one alkylamino_{(C≤12)} group or substituted alkylamino_{(C≤12)} is in its free base form. In some aspects, the kits are for use in treating or preventing a coronavirus infection in a patient.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIGS. 1A-1C: Chloroquine inhibits ATP production. FIG. 1A: Structures of chloroquine (CQ) and hydroxychloroquine (HCQ). FIG. 1B: CQ overnight titration on h293 cells evaluated by CellTiterBlue (measures viability as a function of redox status) and CellTiterGlo (measures viability as a function of ATP levels). FIG. 1C): CQ does not inhibit the luciferase reaction.

FIGS. 2A-2F: Chloroquine inhibits mitochondrial ATP production. FIG. 2A: Short exposure of h293 cells cultured in DMEM to CQ has no apparent effect on ATP levels. FIG. 2B: h293 cells decrease the pH of DMEM media, indicative of ATP production via glycolysis generating lactic acid as a by-product. FIG. 2C: h293 cells can switch between glycolysis and the TCA/ETC in response to metabolic inhibitors. Cells were suspended in the indicated medias (DMEM with glucose, DMEM no glucose, or DMEM no glucose to which glucose was added). Cells were then treated with the glycolysis inhibitor 2-deoxy glucose (2-DG) or the ETC inhibitor rotenone for 1 h, followed by determination of ATP levels using CellTiterGlo. FIG. 2D: h293 cells suspended in DMEM were pre-treated for 1 h with either 2-DG or rotenone to force use of the uninhibited pathway to produce ATP. Cells then exposed to the indicated concentrations of CQ for 2 h, followed by determination of ATP levels using CellTiterGlo. FIG. 2E: Same as FIG. 2D except titrating HCQ. FIG. 2F: Same as FIG. 2D except using Human Diploid Fibroblasts (HDFs) instead of h293s.

FIGS. 3A-3D: Chloroquine inhibits mitochondrial ATP production. FIG. 3A: CQ was titrated on h293 cells in commercially available DMEM media containing amino acids but lacking glucose (lanes 1-6). As a control the titration was repeated after adding glucose to this media (lanes 7-12). FIG. 3B: Order of addition experiments indicated by the schematic below each set. In lanes 7-12 glucose overcomes CQ inhibition. Lanes 13-18 show CQ inhibition is revealed in the presence of 2-DG. FIG. 3C: Using 2-DG and rotenone to identify active metabolic pathways in h293s using L-15 media, which contains galactose instead of glucose (lanes 1-4). Glucose was added to L-15 media in lanes 5-8, which allowed glycolysis and re-established the metabolic switch. FIG. 3D: CQ and HCQ titrations on L-15 media in the absence and presence of glucose.

FIGS. 4A-4G: CQ entry into cells and inhibition of mitochondrial ATP production is pH and time dependent. FIG. 4A: CQ titration on h293 cells cultured in L-15 media lacking NaHCO₃ at 37° C. in a CO₂ incubator. FIG. 4B: L-15 media color and pH change after CO₂ incubation in the absence of NaHCO₃. FIG. 4C: CQ free base and protonated forms. FIG. 4D: Increasing media pH increases ability of CQ to inhibit ATP production. Schematic shows experimental procedure. FIG. 4E: Time course of CQ inhibition. Cells alone or treated with 250 μM CQ for the indicated times and ATP levels analyzed by CellTiterGlo. FIG. 4F: H293 cells were treated with CQ for 1 h and then washed. Nuclear, cytoplasmic, and mitochondrial fractions were generated by dounce homogenization as described in the Methods section, then analyzed for CQ content by UV/Vis. Pure CQ was analyzed to indicate absorption peaks. Cells in the absence of CQ served as the negative control to account for spectra of biomolecules. Glucose was included in the pre-incubation where indicated. FIG. 4G: Red blood cells were pre-incubated with CQ for 1 h. An aliquot was withdrawn to determine ATP levels using CellTiterGlo (inset graph). Remaining cells were washed and lysed by re-suspending in H₂O and vortexing, followed by UV/Vis analysis of the supernatant. Pure CQ was analyzed to show its absorption peaks, while cells alone served as the negative control. The prominent heme peak is indicated by the arrow.

FIGS. 5A-5G: Molecules structurally similar to CQ also inhibit mitochondrial ATP production. FIG. 5A: Quinine structure. FIG. 5B: Quinine titration on h293 cells pre-treated with 2-DG or rotenone to force use of the uninhibited metabolic pathway.

FIG. 5C: Nicotine and diphenhydramine structures. FIG. 5D: Nicotine and diphenhydramine titrations on h293s in the absence and presence 2-DG. FIG. 5E: Effect of cigar smoke on ATP production in A549 lung carcinoma cells. Media was removed and cells washed with PBS. One plate was exposed to cigar smoke for 10 min while the other served as a control. Cells were removed by trypsin and re-suspended in the indicated medias. After incubating for 1 h ATP levels were determined by CellTiterGlo. FIG. 5F: Famotidine structure. FIG. 5G: Famotidine titration on h293s in the absence and presence of 2-DG.

FIG. 6: Model of SARS-CoV-2 infectivity and possible CQ/HCQ anti-viral activities. Plasma membrane and a mitochondrion are depicted. Steps 1-5 propose the initial infection with SARS-CoV-2 occurs in the mitochondrial intermembrane space, which hides the virus from host defenses and provides protons needed for genome release. Eventually ATP synthase activity (red hourglass) is compromised and cells upregulate glycolysis to produce ATP (steps 7 and 8). This acidifies the cytoplasm which provides an environment for increased viral production (steps 9-12). CQ and related molecules could act prophylactically and reduce the probability of initial infection by sequestering protons in the mitochondrial intermembrane space, thereby making this environment less hospitable for the virus (box A). Early post infection CQ could act in a similar manner to slow mitochondrial expansion of the virus (step 5). After the switch to glycolysis, acidification of the cytoplasm could lead to CQ protonation in the cytosol, thereby inhibiting viral replication in this compartment (box B).

### DETAILED DESCRIPTION

Chloroquine (CQ) and hydroxylchloroquine (HCQ) are members of the 4-aminoquinoline class of drugs best known for their anti-malarial activity. More recently, however, controversy has raged regarding their potential as COVID-19 anti-viral therapeutics. Here, CQ and HCQ activity was examined in mammalian tissue culture cells to determine if their purported therapeutic effects might be due to altering host cell pathways. Results indicate the deprotonated form enters the cell in a pH- and time-dependent manner consistent with their lysosomotropic properties. Metabolic analysis showed the drugs accumulate in mitochondria and inhibit ATP production by the TCA cycle and Electron Transport Chain (ETC). This effect is not obvious, however, because cells can switch between glycolysis and oxidative phosphorylation in order to maintain cellular ATP levels. Thus, cells treated with CQ/HCQ alone switch to glycolysis, while cells exposed to the glycolysis inhibitor 2-deoxyglucose (2-DG) alone switch to mitochondrial ATP synthesis. Combining CQ/HCQ with 2-DG had a synergistic effect that dramatically reduced ATP production. In contrast, the ETC inhibitor rotenone forced a switch to glycolysis, and addition of CQ/HCQ had no effect on ATP levels. Cells cultured in media lacking glucose use amino acids to generate ATP, and CQ/HCQ alone inhibited ATP production under these conditions. Finally, cells forced to metabolize galactose, which has to go thru the TCA/ETC, were also very sensitive to CQ/HCQ. CQ/HCQ inhibition of mitochondrial ATP production could be alleviated by glucose addition and activation of glycolysis. These results suggest CQ/HCQ becomes protonated and trapped in the acidic mitochondrial intermembrane space, which inhibits ATP synthesis by sequestering protons needed to drive ATP synthase. Nicotine appeared to function in a similar manner, which might help explain why smokers appear less susceptible to COVID-19. Structurally similar compounds (e.g., diphenhydramine) also specifically inhibited mitochondrial ATP production, suggesting a common mechanism might underlie a subset of potential SARS-CoV-2 anti-viral agents. Mitochondrial accumulation of these drugs and inhibition of ATP synthesis implicate this organelle in COVID-19.

The data provided herein have demonstrated that CQ/HCQ, like SARS-CoV-2, targets the mitochondria and utilizes protons. By identifying the mechanisms whereby CQ/HCQ function to inhibit the progression of the virus, additional compounds were isolated that can be substituted for CQ/HCQ with fewer potential side effects. Additionally, these data demonstrate, that if taken prophylactically, the various compounds can reduce the probability of mitochondrial infection by sequestering the protons needed by the virus for replication. Similarly, once a viral infection occurs, these same compounds can be used therapeutically to reduce the rate at which the virus can propagate through the mitochondria via the same deprotonation mechanism. If the infection progresses and the cells are becoming compromised, these compounds will not be able to fully negate the viral proliferation if used individually; however, they can be combined with glycolysis inhibitors to specifically target and kill the infected cells. This novel combinatory method is effective because the extent of the viral load and degree of compromise will vary from cell to cell and the therapeutics will further inhibit mitochondrial function while pushing less infected cells into glycolysis. Then the glycolysis inhibitor will specifically target all infected cells, reducing their ATP levels and triggering apoptosis. Uninfected cells are still able to metabolize amino acids as a result of their mitochondria not being completely compromised.

### I. Aspects of the Present Disclosure

CQ and related compounds can accumulate in mitochondria and inhibit ATP production via the TCA/ETC. Since this activity might be relevant to its prophylactic and/or therapeutic effects against COVID-19, it is worthwhile considering what this mechanism might tell us about SARS-CoV-2 infection and proliferation. A prophylactic prevents disease, in this case host cell infection by the SARS-CoV-2 virus. Since CQ modulates the mitochondrial environment, the implication is that mitochondria are the initial target of viral infection.

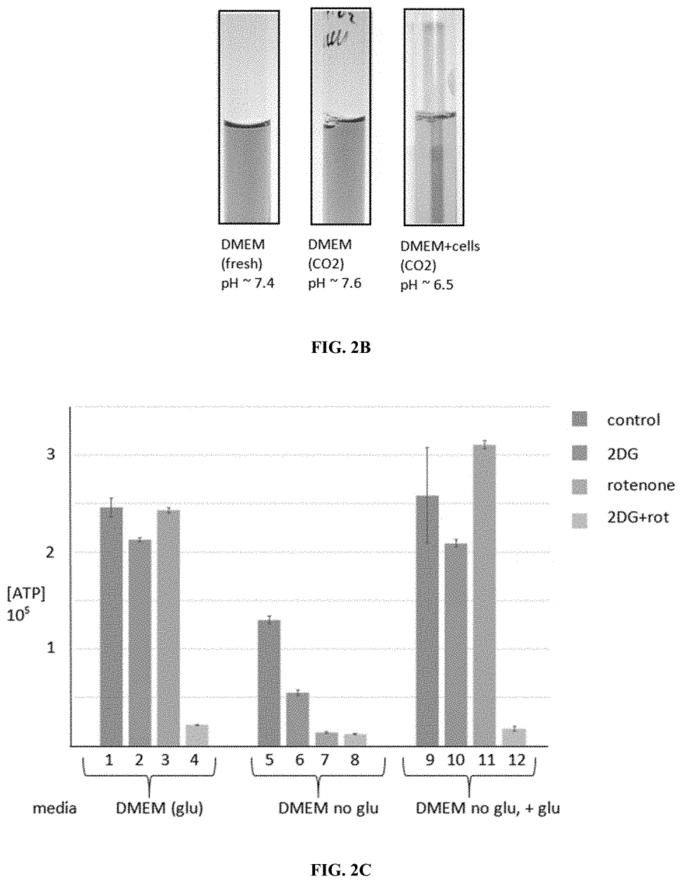

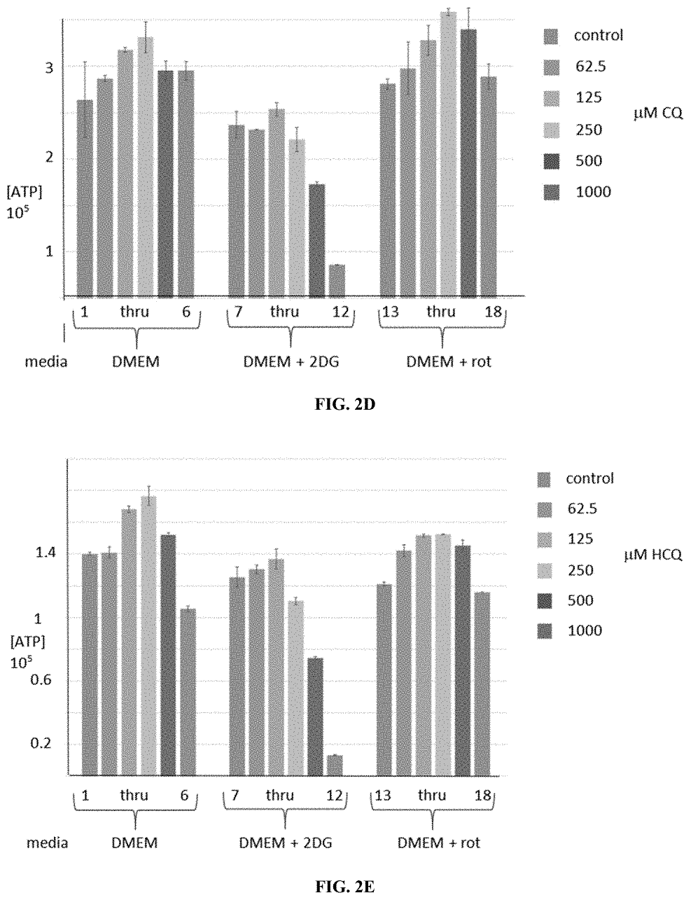

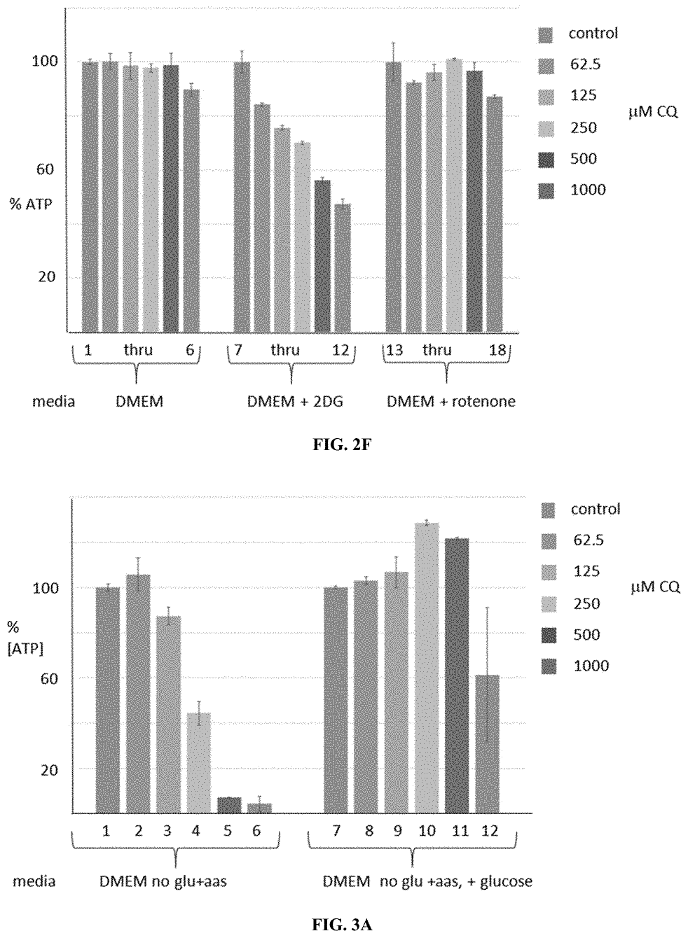

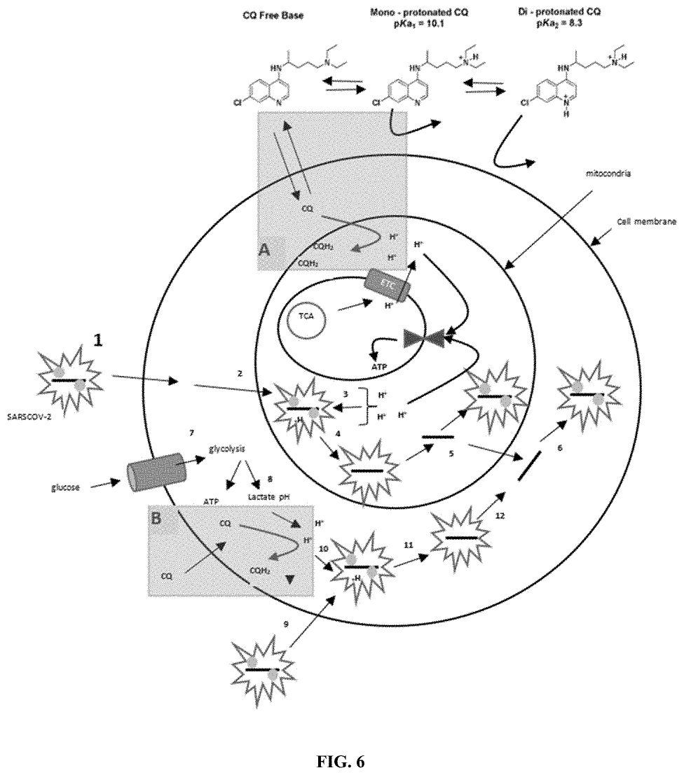

A model of SARS-CoV-2 infectivity and proliferation incorporating this novel step is depicted in FIG. 6. Starting at step 1, the virus enters the cell via well-known mechanisms and forms an endosome. At this point it needs protons to remove capsid proteins and release its RNA genome. Where to get them? The cytosol is somewhat basic and so protons are in short supply, but they are plentiful in the mitochondrial intermembrane space. Thus, the viral endosome may fuse with a mitochondria (step 2) so that the available protons can be used to release the viral genome (steps 3-5). Note that entering mitochondria has the added benefit of hiding the virus and avoiding host cell defenses. At this point the virus has several options. The genome could exit the mitochondria to initiate virus production in the cytoplasm (step 6), but protons are still in short supply, and it might be detected by host cell defenses. A more insidious option would be to generate additional virus in the safe confines of the organelle using mitochondrial ribosomes. This process will eventually deplete enough protons such that the intermembrane space is less conducive to viral proliferation, and ATP synthesis is compromised. At this point there are two options: the virus could move into the cytoplasm, but here again lack of protons and host defenses are a problem. Another possibility is endosomal encapsulation and infection of another mitochondria, followed by a repeat of the above process. When sufficient mitochondria are compromised cellular ATP levels will fall, which as seen in FIG. 2C can cause a switch to glycolysis (step 7). As a consequence, lactic acid is produced (step 8), lowering cytosolic pH and creating a hospitable environment for massive virus production in the cytoplasm (steps 9-12). Furthermore, a switch to glycolysis is analogous to the Warburg effect in cancer, which is thought to reprogram metabolic pathways and direct glucose carbons to increased biomass synthesis (in this case of virus) rather than ATP production.

Viewing this proposed mechanism in the broader context of COVID-19 infection and disease progression, one could envision the mitochondrial infections as a latent stage (relatively asymptomatic), and that of the cytoplasmic infection as acute (leading to extensive viral proliferation, release of viral particles to infect other cells, and host cell damage). Indeed, the well documented decline in mitochondrial function with age could help explain the strong positive correlation between age and rate of SARS-CoV-2 infection/severity. The mitochondrial pool in young people adapts to increased energy requirements or partial mitochondrial inhibition by replicating rapidly, resulting in more mitochondria producing more ATP. This ability decreases as a function of age. Thus, SARS-CoV-2 infection of mitochondria in the young could be compensated for by generating new mitochondria that effectively extend the latent period, perhaps indefinitely. As individuals age and this protective response decreases, the probability that the disease will progress to the acute phase increases. Consistent with this view, the virus appears to be relatively asymptomatic in the original carrier of SARS-CoV-2, an as-yet unknown bat, which as a flying mammal has an extremely high numbers of very active mitochondria. The ability of SARS-CoV-2 to activate a switch to glycolysis by inhibiting mitochondrial ATP production would also help explain the increased rate of infection and disease severity associated with obesity and diabetes. Glycolysis only generates 2 ATP/glucose molecule, as opposed to 34 ATP/glucose molecule when it is completely oxidized in the mitochondria. Thus, as is observed in cancer cells preferentially utilizing aerobic glycolysis (Warburg effect), they tend to require and consume much greater amounts of glucose. Higher blood glucose levels is the defining characteristic of type II diabetes and is often present in obesity as a result of over nutrition upregulating metabolic pathways. As a consequence, the mitochondrial environment might be more conducive to infection and/or the transition to cytoplasmic viral production might occur more rapidly. Elevated glucose levels also provide additional carbon needed for synthesis of biomass, which in the case of COVID-19 would accelerate virus production.

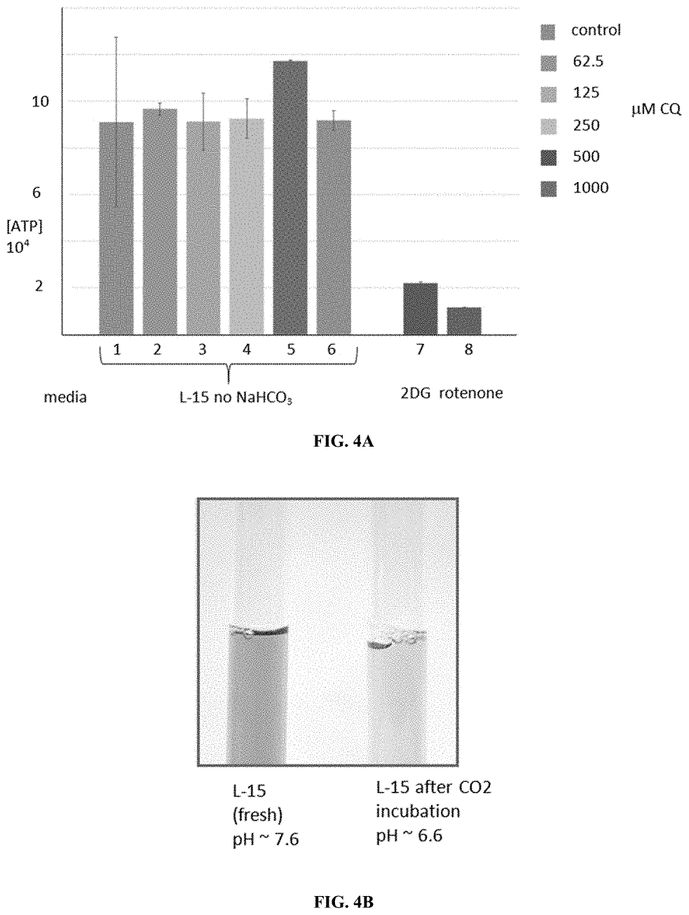

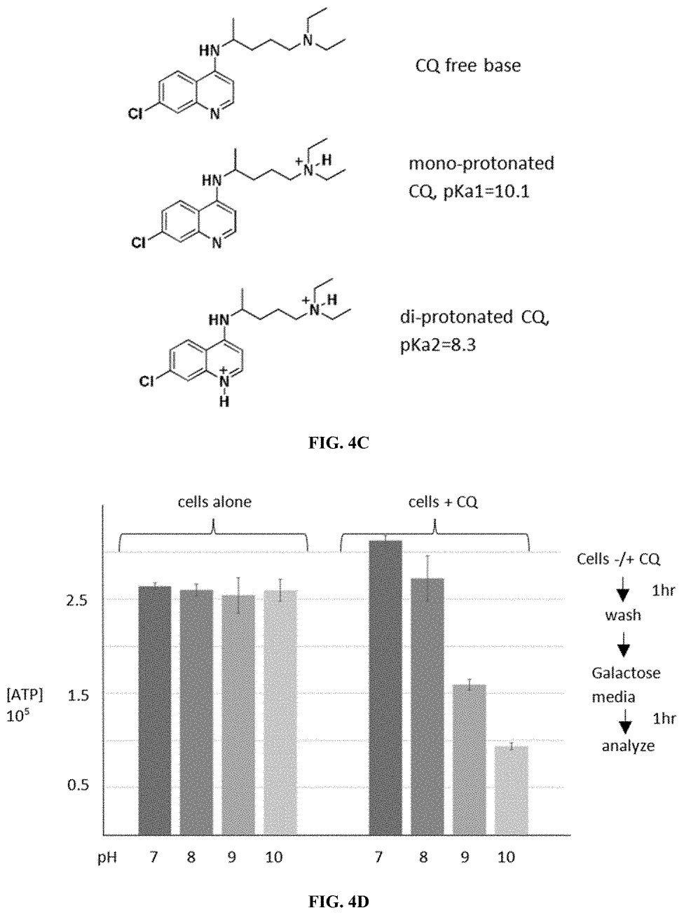

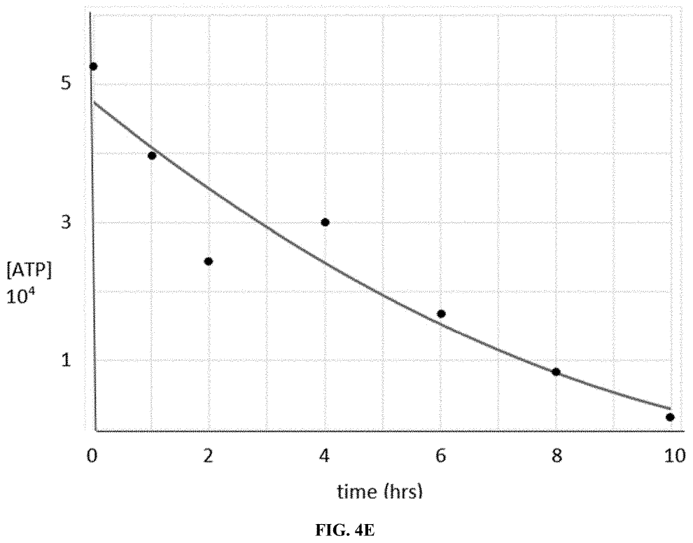

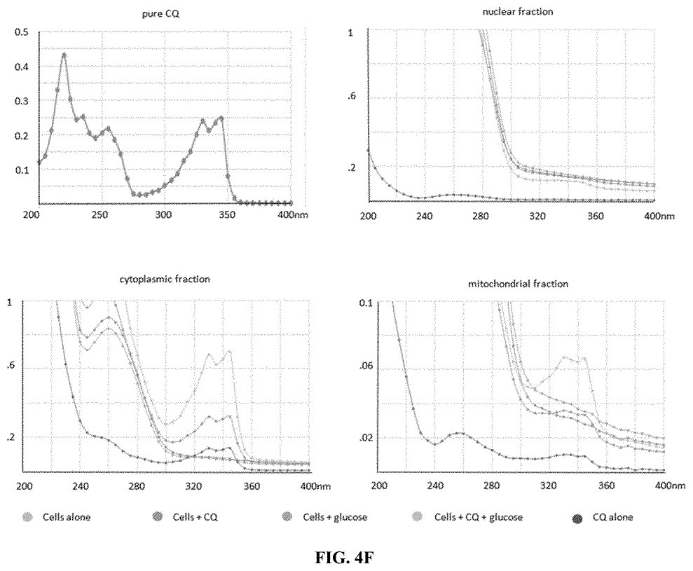

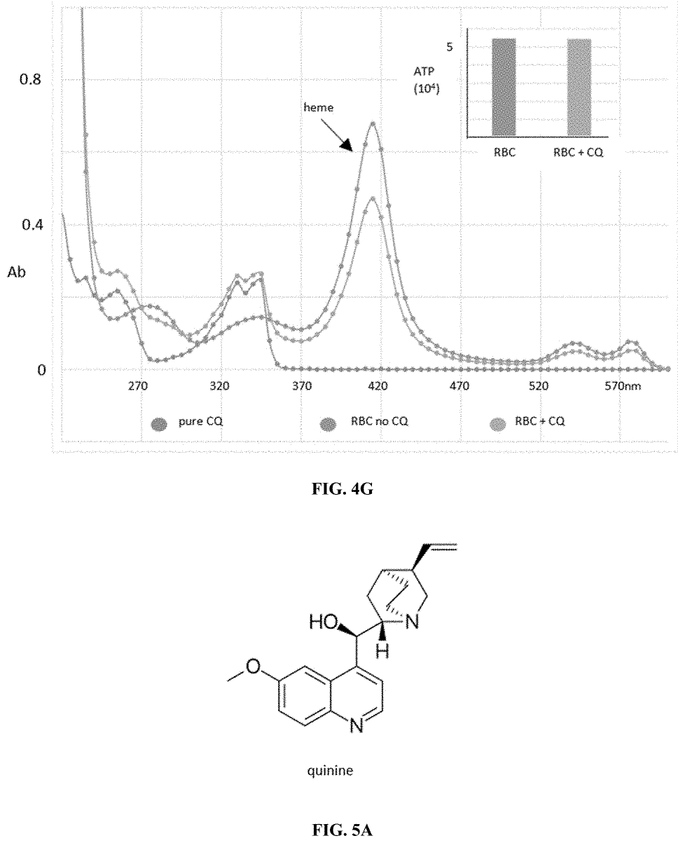

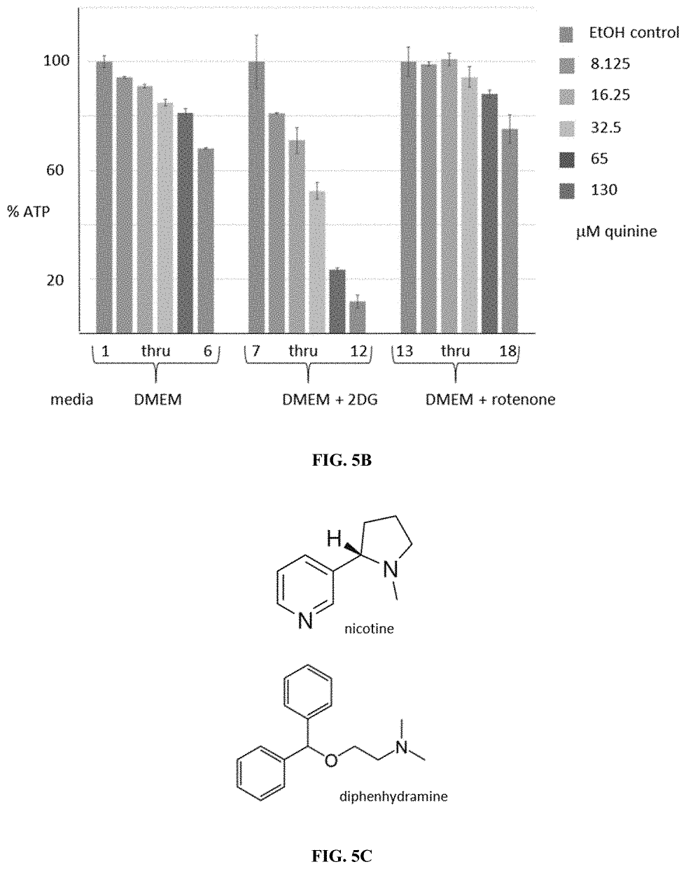

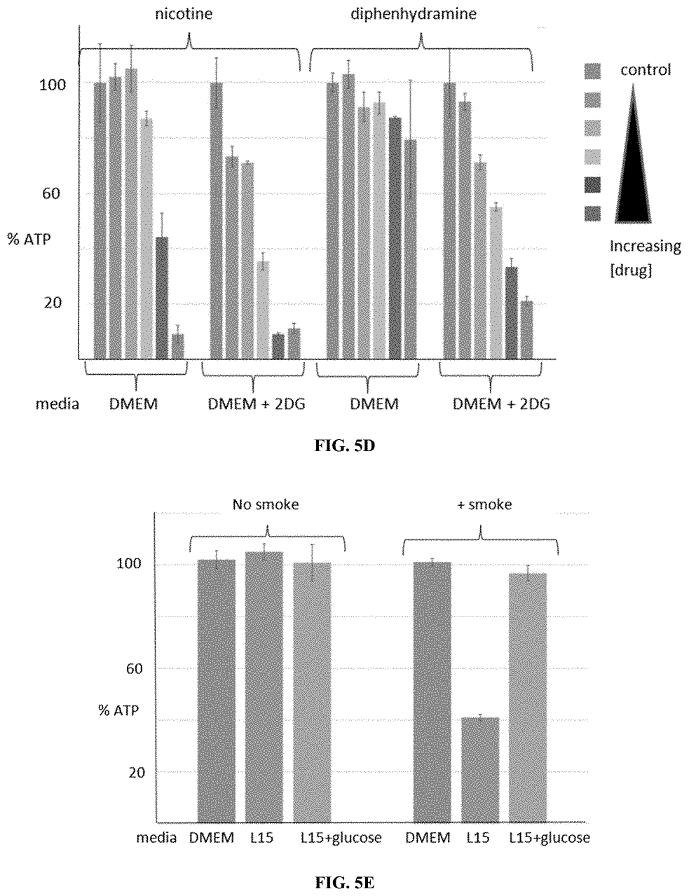

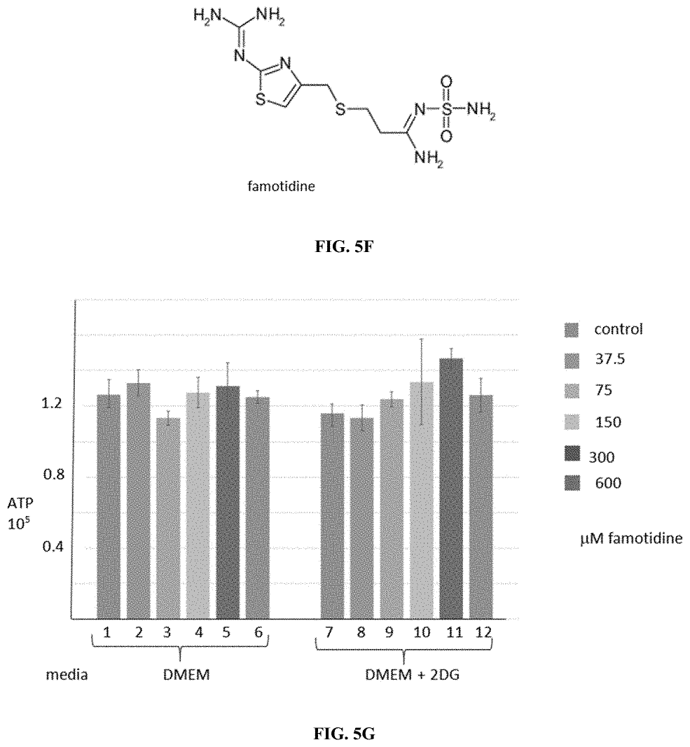

The potential prophylactic and therapeutic effects of CQ and related compounds are shown in the shaded boxes. These drugs could act prophylactically by modestly altering acidity of the intermembrane space (box A) such that it is less conducive to the proposed initial viral infection (steps 1-3). This mechanism might explain the curious observation that smokers appear less likely to be infected, as nicotine can function similarly to CQ (see FIG. 5D). Likewise, prophylactic use and modification of the mitochondrial environment would be expected to slow the rate of viral replication and infection of other mitochondria during the proposed latent stage, thereby retarding disease progression. An additional possibility is based on the observation that glycolysis itself is sufficient to create an acidic environment trapping CQ in the cytoplasm (see FIG. 5C). Thus, CQ protonation could potentially inhibit the proposed acute phase in the cytoplasm (FIG. 6, steps 9-12) as well. In advanced stages of COVID-19, however, the large number of infected cells forced to use glycolysis might result in acidification of surrounding tissue due to the excretion of large amounts of lactate, thereby causing extracellular protonation of CQ and inhibiting its ability to enter infected cells (see FIG. 4D). A complicating factor is that the model also illustrates how higher concentrations of CQ could actually be detrimental, especially if they inhibit mitochondrial ATP production and activate the metabolic switch to glycolysis. This would lower the cytosolic pH and generate a cytoplasmic environment more conducive to viral infection/proliferation. The ability of high CQ levels to inhibit mitochondrial ATP production could also explain its well-known cardiotoxicity. Heart muscle generates most of its ATP from beta oxidation of fatty acids in mitochondria, so CQ inhibition combined with an inability to use glycolysis (or failure of this pathway to generate sufficient ATP), could exacerbate damage to this tissue.

Despite these potential therapeutic mechanisms of CQ, evidence for its use is decidedly mixed and controversial. It may be that a structurally related molecule will show greater efficacy and reduced side effects. For example, various modeling efforts and drug screening against SARS-CoV-2 infection of African green monkey cells have identified a diverse group of potential therapeutics, some of which from this work would be predicted to function like CQ (e.g., camostat, Clemastine, Dextromethorphan). Other commercially available drugs have not been specifically implicated as potential COVID-19 treatments, but based on structural similarity to CQ might be of interest (e.g., chlorpromazine, disulfiram, rosiglitazone, diphenhydramine). An alternative approach would be to focus on relevant host cell pathways manipulated by SARS-CoV-2 infection. Drugs specifically targeting mitochondria are an obvious example, especially if compromised mitochondria could be cleared without affecting host cell function. Another interesting avenue of investigation is based on the proposed metabolic changes that take place in infected cells, in particular the switch to glycolysis (FIG. 6). Since at this point the TCA/ETC is compromised and infected cells are dependent on the glycolytic pathway, it might be possible to specifically target infected cells with a simple glycolysis inhibitor like 2-DG. While normal uninfected cells can switch to the TCA/ETC in the presence of 2-DG and metabolize amino acids (see FIG. 2C), infected cells cannot. The subsequent drop in ATP levels could be sufficient to cause death of infected cells.

The SARS-CoV-2 virus causing COVID-19 has emerged as a serious and ongoing global threat. A biochemical analysis of CQ and HCQ mechanism of action in tissue culture cells suggests these and related drugs might work in part by entering mitochondria and becoming protonated, which depletes H+ needed for ATP production. This mechanism in turn suggests a model of SARS-CoV-2 infection whereby the virus first infects a mitochondria, which not only provides a ready source of protons but also serves as a cloaking device from host defense mechanisms. Subsequent viral expansion through the mitochondrial population (latent phase) could help maintain viral invisibility, while prepping the cytoplasm for massive virus production via a switch to glycolysis that decreases cytosol pH and redirects glucose carbons into viral biomass production (acute phase).

### II. Compounds

The compounds of the present invention (also referred to as “compounds of the present disclosure”) are shown, for example, above, in the summary of the invention section, and in the claims below.

Some of the compounds of the present invention may be generally classified as glycolysis inhibitors. For example, 2-deoxy-D-glucose (2-DG) is a glycolysis inhibitor. Additional examples of glycolysis inhibitors include, without limitation, hexokinase inhibitors (e.g., metformin), glyceraldehyde-3-phosphate dehydrogenase inhibitors (e.g., iodoacetate, koningic acid, 3-bromopyruvate, methylglyoxal), phosphoglycerate inhibitors (e.g., HKB99), pyruvate kinase inhibitors (e.g., shikonin, deoxyshikonin, isobutylshikonin, B,B-dimethylacrylshikonin, isovalerylshikonin, 3 (N-(3-Carboxy-4-hydroxy) phenyl-2,5-dimethylpyrrole), lactate dehydrogenase inhibitors (e.g., galloflavin, FX-11, GNE-140, gossypol, oxamic acid, NHI-1), enolase inhibitors (e.g., AP-111-a4, AP-111-a4 hydrochloride), phosphoglycerate kinase inhibitors (e.g., 1,3-bisphospho-D-glyceric acid and analogues thereof), aldolase inhibitors (e.g., 2-phospahte-naphthalene 6-bisphosphonate, 2-naphthol 6-bisphosphnate, 1-phosphonate-benzyne 4-bisphosphonate), pyruvate dehydrogenase inhibitors (e.g., 6,8-bis(benzylthio)-octanoic acid, dichloroacetate), phosphofructo-2-kinase inhibitors (e.g., 3PO), GLUT (glucose transporter) inhibitors (e.g., phloretin, STF31, WZB117), and multiple pathway inhibitors (e.g., quercetin).

Other compounds of the present invention may be generally classified as having one or more aromatic rings and at least one alkylamino_{(C≤12)} group. Without being bound by any theory, in addition or alternative, these compounds may function as inhibitors of oxidative phosphorylation by inhibiting mitochondrial ATP synthesis by sequestering protons needed to drive ATP synthase. Examples of such compounds include, without limitation, compounds having the following CAS IDs: 51-43-4 ((−)-epinephrine); 150-05-0 (D-(+)-epinephrine); 329-65-7 (DL-adrenaline); 52134-32-4 (d-epinephrine hydrochloride); 59-42-7 (phenylephrine); 7683-59-2 (isoproterenol); 709-55-7 (etilefrine); 94-07-5 ((±)-synephrine); 59721-29-8 (camostat); 10253-82-4 (butylnoradrenaline); 108873-53-6 (N-butylepinephrine); 14638-70-1 ((+)-isoproterenol); 1477-63-0 ((±)-phenylephrine); 7683-59-2 (isoprenaline); 154-86-9 (DL-phenylephrine hydrochloride)); 2947-00-4 (ethyladrenaline); 2964-04-7 ((+)-isoproterenol); 3571-71-9 (metaterol); 3703-79-5 (bamethan); 42145-91-5 (etilefrine pivalate hydrochloride); 4232-09-1 (ethylnorsynephrine); 4232-10-4 (p-hydroxy-alpha-(propylaminomethyl)-benzyl alcohol); 51-31-0 ((R)-(−)-isoproterenol); 532-80-9 ((+)-synephrine); 5716-20-1 (bamethan sulfate); 586-06-1 (orciprenaline); 5985-28-4 (synephrine hydrochloride); 614-35-7 ((−)-synephrine); 64037-41-8 (1-(3,4-Dihydroxyphenyl)-2-methylamino-1-butanol hydrochloride); 64037-63-4 (N-butylepinephrine); 6422-43-1 (1-(3,4-Dihydroxyphenyl)-2-ethylaminoethanol hydrochloride); 6924-25-0 (WIN 5595); 7376-69-4 (propyladrenaline); 943-17-9 (etilefrine hydrochloride); 114-45-4 (isoproterenol sulfate); 299-95-6 (isoprenaline sulfate); 54750-10-6 ((−)-isoproterenol (+)-bitartrate salt); 67-04-9 (synephrine tartrate); 949-36-0 (isoproterenol hydrochloride); 7279-75-6 (isoetharine mesylate); 118-42-3 (hydroxychloroquine); 54-05-7 (chloroquine); 50-63-5 (chloroquine phosphate); 132-73-0 (chloroquine sulfate); 1446-17-9 (chloroquine phosphate); 85-10-9 (3-methylchloroquine); 97-77-8 (disulfiram); 1634-02-2 (tetrabutylthiuram disulfide); 2556-42-5 (tetrapropylthiuram disulphide); 22718-30-5; 42542-10-(3,4-methylenedioxymethamphetamine); 64057-70-1 (N-Methyl-3,4-methylenedioxyamphetamine Hydrochloride); 14089-52-2 (3,4-methylenedioxyethamphetamine); 147-24-0 (diphenhydramine hydrochloride); 58-73-1 (diphenhydramine); 31065-89-1 ([2-(Diphenylmethoxy)ethyl]trimethylammonium bromide); 341-69-5 (orphenadrine hydrochloride); 83-98-7 (orphenadrine); 17630-53-4 (orphenadrine); 18483-57-3 (N,N-Dimethyl-2-(di-(p-tolyl)methoxy)ethylamine hydrochloride); 2827-06-7 (2-[2-[bis(2,6-dimethylphenyl)methoxy]ethoxy]ethyl-dimethylazaniumchloride); 35471-87-5 (diethylaminopropyl benzhydryl ether hydrochloride); 4024-34-4 (p-methyl diphenhydramine hydrochloride); 4724-58-7 (2-Dimethylaminoethyl-2-methyl-benzhydryl ether citrate); 53499-34-6 (N,N-Diethyl-2-(phenyl-(p-tolyl)methoxy)ethylamine hydrochloride); 63918-12-7 (N,N-Dimethyl-2-(di-(o,m′-tolyl)methoxy)ethylamine hydrochloride); 63918-13-8 (N,N-Dimethyl-2-(di-(o,p′-tolyl)methoxy)ethylamine hydrochloride); 63918-14-9 (N,N-Dimethyl-2-(mesityl(phenyl)methoxy)ethyl amine hydrochloride); 63918-17-2 (N,N-dimethyl-2-(phenyl(4-propylphenyl)methoxy)ethan-1-amine hydrochloride); 63918-24-1 (2-(p-Isopropyldiphenylmethoxy)-N,N-dimethylethylamine hydrochloride); 63978-56-3 (beta-(beta-Diethylaminoethoxy)ethyl benzhydryl ether); 642-58-0 (ethylbenzhydramine); 1600-19-7 (xyloxemine); 19804-27-4 (toladryl); 2820-51-1 (nicotine hydrochloride); 25162-00-9 (R-(+)-nicotine); 21361-93-3 (nicotine hydrochloride); 21446-46-8 (Nicotine, methiodide (monomethyl)); 22083-74-5 (0-nicotine); 54-11-5 ((−)-nicotine); 5959-86-4 (N-Methylnicotinium iodide); 6019-02-9 (nicotine dihydrochloride); 23950-04-1 (alpha-Nicotine); 5979-92-0 (ethylnornicotine); 83-79-4 (rotenone); 70288-86-7 (ivermectin); 123-75-1 (pyrrolidine); 1420477-60-6 (acalabrutinib); 76824-35-6 (famotidine); 137-58-6 (lidocaine); 130-95-0 (quinine); 616-45-5 (2-pyrrolidone); 50-53-3 (chlorpromazine); 122320-73-4 (rosiglitazone); 147-24-0 (diphenhydramine hydrochloride); 259793-96-9 (favipiravir); 17-7-4; 125-71-3 (dextromethorphan); 60-82-2 (phloretin).

All the compounds of the present invention may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all the compounds of the present invention are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the compounds of the present invention have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

Compounds of the present invention may contain one or more asymmetrically substituted carbon or nitrogen atom and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S or the R configuration. In some embodiments, the present compounds may contain two or more atoms which have a defined stereochemical orientation.

Chemical formulas used to represent compounds of the present invention will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In some embodiments, compounds of the present invention function as prodrugs or can be derivatized to function as prodrugs. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a patient, cleaves to form a hydroxy, amino, or carboxylic acid, respectively.

In some embodiments, compounds of the present invention exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will be appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the compounds provided herein, including any solvates thereof are within the scope of the present invention.

### III. Combination Therapy

The combination therapies of the present invention may also find use in further combinations. Effective combination therapy may be achieved with a single composition or pharmacological formulation that includes multiple agents, or with multiple compositions or formulations, administered at the same time, wherein one composition includes a combination described elsewhere herein, and the other includes the second agent(s). Alternatively, the therapy may precede or follow the other agent treatment by intervals ranging from minutes to months.

Various combinations may be employed, such as when a combination described elsewhere herein is “A” and “B” represents a secondary agent, non-limiting examples of which are described below:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

It is contemplated that other therapeutic agents may be used in conjunction with the treatments of the current invention. In some embodiments, the present invention contemplates the use of one or more other therapies for the treatment of COVID-19 include the use of a SARS-CoV-2 protease inhibitor, anti-platelet drugs, an anti-coagulation agent, a human type I interferon, a corticosteroid, or remdesivir.

In some embodiments, the anti-platelet drug is aspirin, an ADP receptor antagonist (e.g., ticlopidine, clopidogrel, cangrelor, prasugrel, ticagrelor, thienopyridine), or a glycoprotein IIb/IIIa receptor inhibitor (e.g., abciximab, eptifibatide, ticofiban). In some embodiment, the anti-coagulation agent is rivaroxaban, apixaban, dipyridamole, cilostazol, atromentin, edoxaban, fondaprinux, betrixaban, letaxaban, eribaxaban, hirudin, a thrombin inhibitor (e.g., lepirudin, desirudin, dabigatran, bivalirudin, ximelagatran), argatroban, batroxobin, hementin, low molecular weight heparin, unfractionated heparin, vitamin E, or a vitamin K antagonist (e.g., warfarin (Coumadin), acenocoumarol, phenprocoumon, phenindione).

Human type I interferons (IFNs) are a large subgroup of interferon proteins that help regulate the activity of the immune system. The mammalian types are designated IFN-α (alpha), IFN-β (beta), IFN-κ (kappa), IFN-δ (delta), IFN-ε (epsilon), IFN-τ (tau), IFN-ω (omega), and IFN-ζ (zeta, also known as limitin). Type I interferons have shown efficacy against the replication of various viruses, included Zika virus, chikungunya virus, flaviviruses, and hepatitis C virus. “Interferon compounds” include interferon-alpha, interferon-alpha analogues, interferon-alpha derivatives, interferon-alpha conjugates, interferon beta, interferon-beta analogues, interferon-beta derivatives, interferon-beta conjugates and mixtures thereof. The whole protein or its fragments can be fused with other peptides and proteins such as immunoglobulins and other cytokines. Interferon-alpha and interferon-beta conjugates may represent, for example, a composition comprising interferon-beta coupled to a non-naturally occurring polymer comprising a polyalkylene glycol moiety. Preferred interferon compounds include Roferon®, Intron®, Alferon®, Infergen®, Omniferon®, Alfacon-1, interferon-alpha, interferon-alpha analogues, pegylated interferon-alpha, polymerized interferon-alpha, dimerized interferon-alpha, interferon-alpha conjugated to carriers, interferon-alpha as oral inhalant, interferon-alpha as injectable compositions, interferon-alpha as a topical composition, Roferon® analogues, Intron® analogues, Alferon® analogues, and Infergen® analogues, Omniferon® analogues, Alfacon-1 analogues, interferon beta, Avonex™, Betaseron™, Betaferon™, Rebif™, interferon-beta analogues, pegylated interferon-beta, polymerized interferon-beta, dimerized interferon-beta, interferon-beta conjugated to carriers, interferon-beta as oral inhalant, interferon-beta as an injectable composition, interferon-beta as a topical composition, Avonex™ analogues, Betaseron™, Betaferon™ analogues, and Rebif™ analogues. Alternatively, agents that induce interferon-alpha or interferon-beta production or mimic the action of interferon-alpha or interferon-beta may also be employed. Interferon inducers include tilorone, poly(I)-poly(C), imiquimod, cridanimod, bropirimine.

It is contemplated that other agents may be used in combination with certain aspects of the present invention to improve the therapeutic efficacy of treatment. These additional agents include anti-virals, corticosteroids (e.g., dexamethasone), chloroquine, hydroxychloroquine, remdesivir, favipiravir, lopinavir, and ritonavir.

### IV. Pharmaceutical Formulations and Routes of Administration

In another aspect, for administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of a compound disclosed herein formulated with one or more excipients and/or drug carriers appropriate to the indicated route of administration. In some embodiments, the compounds disclosed herein are formulated in a manner amenable for the treatment of human and/or veterinary patients. In some embodiments, formulation comprises admixing or combining one or more of the compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the compounds may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Pharmaceutical formulations may be administered by a variety of methods, e.g., orally or by injection (e.g. subcutaneous, intravenous, and intraperitoneal). Depending on the route of administration, the compounds disclosed herein may be coated in a material to protect the compound from the action of acids and other natural conditions which may inactivate the compound. To administer the active compound by other than parenteral administration, it may be necessary to coat the compound with, or co-administer the compound with, a material to prevent its inactivation. In some embodiments, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The compounds disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The compounds disclosed herein can be administered orally, for example, with an inert diluent or an assimilable edible carrier. The compounds and other ingredients may also be enclosed in a hard or soft-shell gelatin capsule, compressed into tablets, or incorporated directly into the patient's diet. For oral therapeutic administration, the compounds disclosed herein may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such pharmaceutical formulations is such that a suitable dosage will be obtained.

The therapeutic compound may also be administered topically to the skin, eye, ear, or mucosal membranes. Administration of the therapeutic compound topically may include formulations of the compounds as a topical solution, lotion, cream, ointment, gel, foam, transdermal patch, or tincture. When the therapeutic compound is formulated for topical administration, the compound may be combined with one or more agents that increase the permeability of the compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the therapeutic compound to reach the posterior portion of the eye. Ophthalmic topical administration can be formulated as a solution, suspension, ointment, gel, or emulsion. Finally, topical administration may also include administration to the mucosa membranes such as the inside of the mouth. Such administration can be directly to a particular location within the mucosal membrane such as a tooth, a sore, or an ulcer. Alternatively, if local delivery to the lungs is desired the therapeutic compound may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical carrier. In some embodiments, the specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. In some embodiments, active compounds are administered at a therapeutically effective dosage sufficient to treat a condition associated with a condition in a patient. For example, the efficacy of a compound can be evaluated in an animal model system that may be predictive of efficacy in treating the disease in a human or another animal.

In some embodiments, the effective dose range for the therapeutic compound can be extrapolated from effective doses determined in animal studies for a variety of different animals. In some embodiments, the human equivalent dose (HED) in mg/kg can be calculated in accordance with the following formula (see, e.g., Reagan-Shaw et al., FASEB 22(3):659-661, 2008, which is incorporated herein by reference):

HED (mg/kg)=Animal dose (mg/kg)×(Animal Kₘ/Human Kₘ)

Use of the Kₘ factors in conversion results in HED values based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present disclosure or composition comprising a compound of the present disclosure administered to a patient may be determined by physical and physiological factors such as type of animal treated, age, sex, body weight, severity of condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

In some embodiments, the amount of the active compound in the pharmaceutical formulation is from about 2 to about 75 weight percent. In some of these embodiments, the amount if from about 25 to about 60 weight percent.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The agent(s) may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may be taken orally and that the timing of which is or is not dependent upon food intake. Thus, for example, the agent can be taken every morning and/or every evening, regardless of when the patient has eaten or will eat.

### V. Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanyl” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “thiocarbonyl” means —C(═S)—; “sulfonyl” means —S(O)₂—; and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, the formula

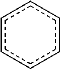

covers, for example,

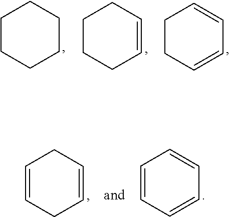

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g., [symbol] for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

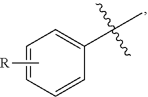

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

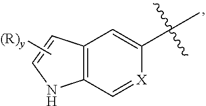

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “Cn” defines the maximum number (n) of carbon atoms that can be in the group/class, with the minimum number as small as possible for the group/class in question. For example, it is understood that the minimum number of carbon atoms in the groups “alkyl_{(C≤8)}”, “alkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C₁₋₄-alkyl”, “C₁₋₄-alkyl”, “alkyl_{(C1-4)}”, and “alkyl_{(C≤4)}” are all synonymous. Except as noted below, every carbon atom is counted to determine whether the group or compound falls with the specified number of carbon atoms. For example, the group dihexylamino is an example of a dialkylamino_{(C12)} group; however, it is not an example of a dialkylamino(co group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic π system. An aromatic compound or chemical group may be depicted as a single resonance structure; however, depiction of one resonance structure is taken to also refer to any other resonance structure. For example:

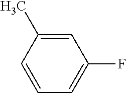

is also taken to refer to

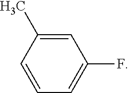

Aromatic compounds may also be depicted using a circle to represent the delocalized nature of the electrons in the fully conjugated cyclic π system, two non-limiting examples of which are shown below:

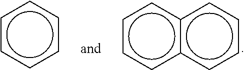

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

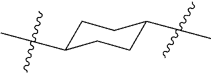 [SMILES: *C1CCC(*)CC1]

is a non-limiting example of cycloalkanediyl group. A “cycloalkane” refers to the class of compounds having the formula H—R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “alkynyl” refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

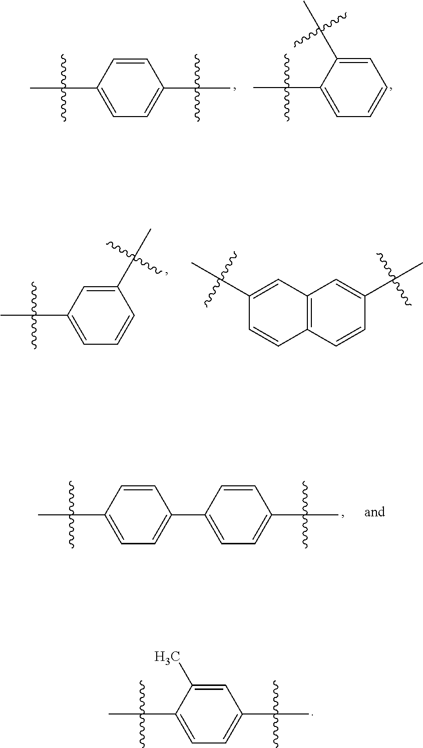

An “arene” refers to the class of compounds having the formula H—R, wherein R is aryl as that term is defined above. Benzene and toluene are non-limiting examples of arenes.

The term “aralkyl” refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl, isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes.

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings are fused. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group.

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group.

The term “alkoxy” refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The term “amido” (acylamino), when used without the “substituted” modifier, refers to the group —NHR, in which R is acyl, as that term is defined above. A non-limiting example of an amido group is —NHC(O)CH₃.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, the variation that exists among the study subjects or patients, or a value that is within 10% of a stated value.

An “active ingredient” (AI) or active pharmaceutical ingredient (API) (also referred to as an active compound, active substance, active agent, pharmaceutical agent, agent, biologically active molecule, or a therapeutic compound) is the ingredient in a pharmaceutical drug that is biologically active.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to the patient or subject, is sufficient to effect such treatment or prevention of the disease as those terms are defined below.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an active pharmaceutical ingredient of the present invention. The prodrug itself may or may not have activity with in its prodrug form. For example, a compound comprising a hydroxy group may be administered as an ester that is converted by hydrolysis in vivo to the hydroxy compound. Non-limiting examples of suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, and esters of amino acids. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2′, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### VI. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Materials and Methods

Drugs. All drugs were obtained from Sigma except diphenhydramine (purchased commercially as 25 mg allergy tablets) and famotidine (purchased commercially as 10 mg acid controller tablets). All compounds were dissolved in ultrapure water except quinine, which was re-suspended in ethanol. Rotenone was prepared as a concentrated solution in 100% DMSO, then diluted to a working stock (125 μM) in water (final concentration 2.5 μM). 2-DG was used at a final concentration of 20 mM. Diphenhydramine and famotidine were crushed by mortar and pestle, re-suspended in H₂O and rotated at room temperature for 1 h. Insoluble material (e.g., cellulose) was removed by centrifugation. Drug presence was confirmed by UV/Vis analysis. Cigar smoke was generated from commercially available Backwoods brand containing nicotine.

Cell culture. The following immortalized cells were obtained from the American Tissue Culture Collection: H293 kidney epithelial cells, HDFs (human diploid fibroblasts immortalized by telomerase expression), and U549 lung carcinoma cells. Cells were maintained in DMEM (Fisher Scientific) supplemented with 10% fetal bovine serum (FBS; Atlas Biologicals, Fort Collins, Colo.). DMEM media contained high glucose (25 mM) and the standard amino acids as carbon sources. Cultures were maintained in a 37° C. water jacketed incubator with 5% CO₂. When needed cells were removed from the plate using trypsin. It is important to note that FBS and many commercial preparations of trypsin contain glucose. Unless indicated otherwise, after removal, cells were pelleted by centrifugation (2K 4 min room temperature), aspirated, and re-suspended in the desired media. Other media used in this study were DMEM no glu (Fisher Scientific), which is identical to DMEM except it lacks glucose. L-15 media (Fisher Scientific) contains amino acids but replaces glucose with 5 mM galactose. It also lacks sodium bicarbonate (NaHCO₃), which was added to the same concentration as in DMEM (44 mM) in all experiments except FIGS. 4A and 4B. Its presence is essential to buffer the media and prevent its acidification in the CO₂ incubator. A DMEM type media lacking sodium bicarbonate was also generated using individual components obtained from Fisher Scientific and Sigma. This media was used for FIG. 4D, where its pH was varied by the addition of 500 mM Hepes pH 7-10 (25 mM final concentration).

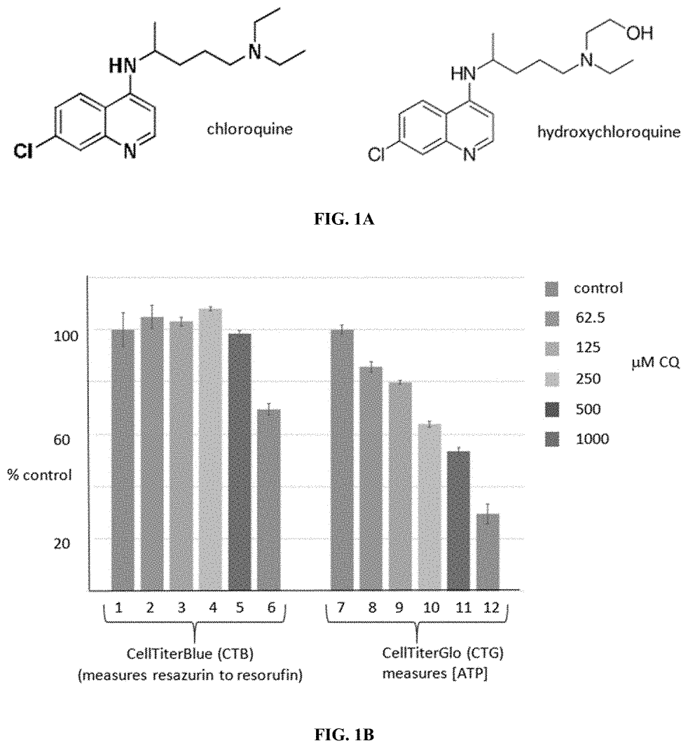

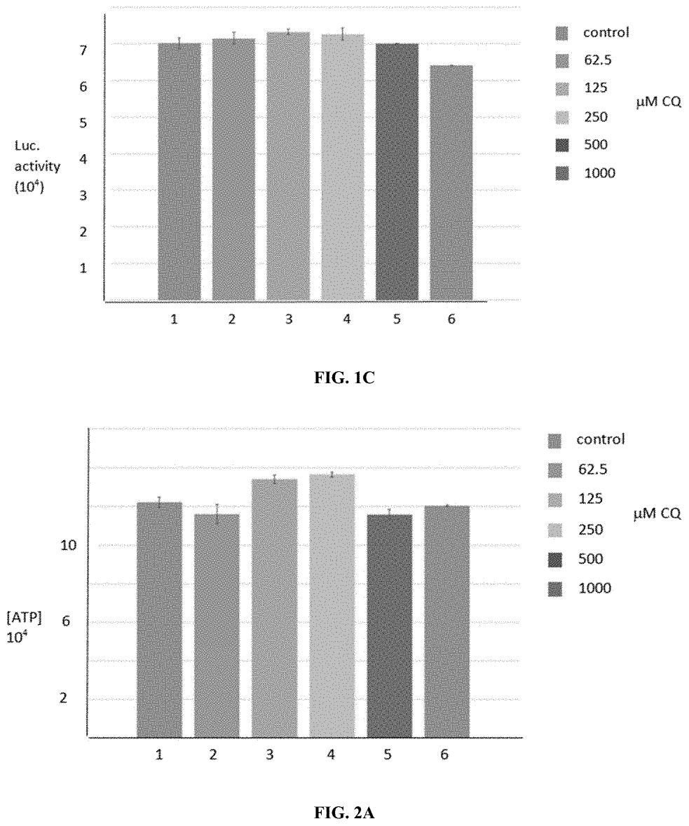

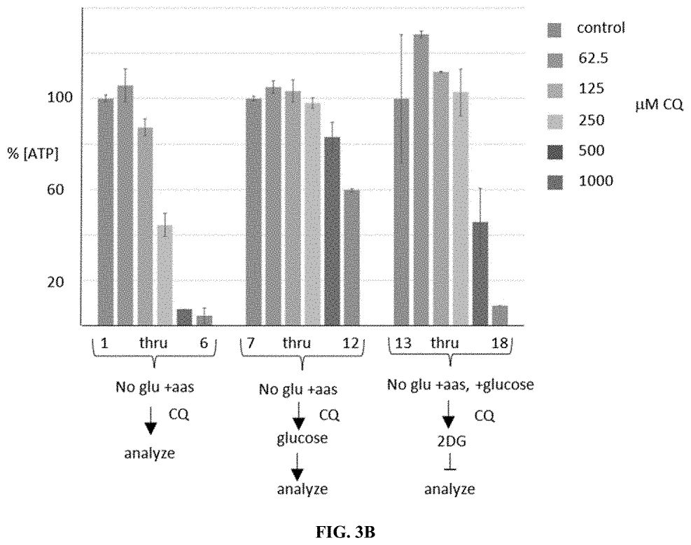

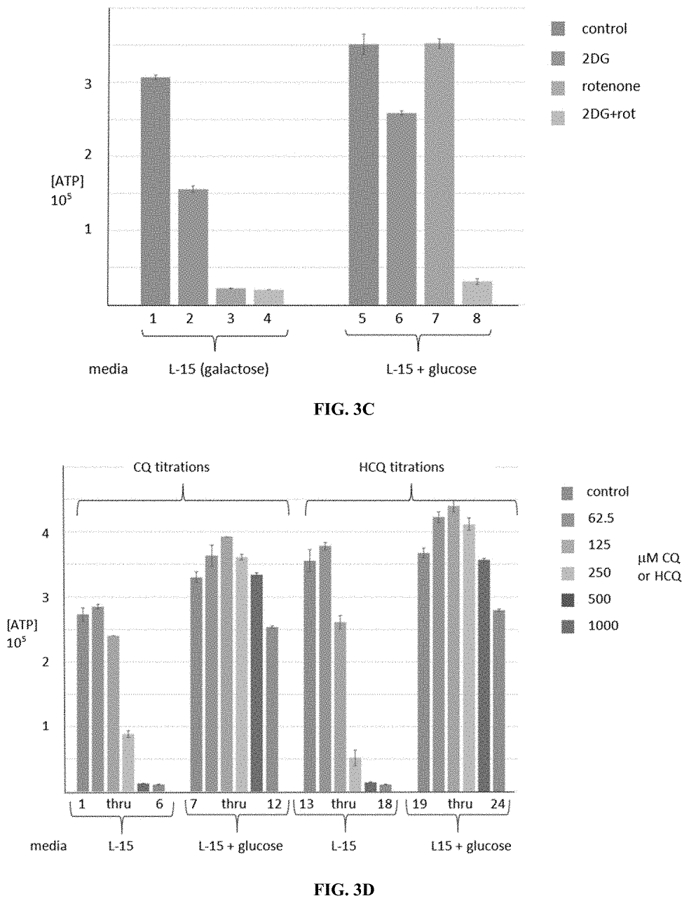

Inhibition assays. In general, short term immediate effects of the indicated drugs on ATP levels were analyzed by removing growing cells from a p100 mm plate using trypsin, washing with PBS, and re-suspending in the indicated media. After counting using a hemocytometer, 25-50k cells in 100 mL media were distributed in a 96 well white flat bottom, non-treated polystyrene assay plate. In some cases, media was supplemented by the addition of glucose (20 mM final). The plate was typically incubated for 30 min at 37° C. in a CO₂ incubator to allow metabolic pathways to adapt to the media. Drugs when then added in the order and at the concentration indicated for each figure, followed by shaking (700 RPM 10 sec), and returned to the incubator. Incubation times were generally between 1 and 2 h so that direct and immediate effects on ATP production could be evaluated. In most cases samples were run in duplicate or triplicate and the standard deviation determined. FIG. 1B is a more traditional drug analysis, in that cells were seeded in a tissue culture treated 96-well plate, allowed to attach overnight, then treated with 10 μL CellTiterBlue resazurin reagent (Promega) to determine effects on viability. Plates were shaken and incubated in the dark for 1-4 h, followed by excitation at 560 nm and determination of fluorescence emitted at 590 nm using a Tecan Safire plate reader. Because CellTiterBlue does not lyse cells, it was possible to add 10 μL CellTiterGlo to the same plate, shake 5 min, and determine ATP levels by using a photoluminomter (BioTek Cytation 5) to measure luminescence. Luciferase activity alone (FIG. 1C) was measured by adding 10 CellTiterGlo reagent to 100 μL DMEM media in the absence of cells. CQ was then added and the reaction initiated by the addition of 5 μL ATP. The luminescence was determined immediately and at later times as described above. For the order of addition experiment in FIG. 3B, cells were pre-incubated with CQ for 2 h, followed by the indicated additions for an additional 1 h.

Fractionation. The published protocol of Clayton and Shadel was used with slight modifications. Briefly, 2.5×10⁶ h293 cells were removed from plates and re-suspended in 1 mL L1-5 media without and with 20 mM glucose. CQ in the absence of cells served as a negative control. Tubes were rotated 2 h room temperature to allow cellular entry of CQ. Cells were then pelleted by centrifugation, washed using L-15 media, and pelleted again. The cell pellet was re-suspended in 1 mL ice-cold RSB hypotonic buffer and transferred to a cold 3 mL dounce homogenizer. After allowing cells to swell for 10 min (on ice), the cold B pestle was used to break them open (10 strokes). Homogenization was confirmed by phase contrast microscopy. 0.7 mL MS homogenization buffer was then added and the solution centrifuged at 1300 g for 5 min at 4° C. to remove nuclei (nuclear fraction). The supernatant was then centrifuged at 10,000 g for 15 min generate a cytoplasmic fraction (supernatent) and a mitochondrial fraction (pellet). The nuclear and mitochondrial pellets were re-suspended in 750 μL H₂O, sonicated, and pelleted to remove insoluble material. The resulting cellular fractionations were analyzed directly for the presence of CQ by UV/Vis. Spectra were compared to that of purified CQ.

For the red blood cell analysis, ˜50 μL cells were obtained from a finger prick and immediately added to PBS with 5 mM EDTA to prevent coagulation. Cells were then pelleted and re-suspended in DMEM containing 5 mM glucose (physiological concentration). After 2 h incubation at room temperature rotating, cells were pelleted, washed in PBS, and lysed in 750 μL H₂O with vortexing. After centrifugation to remove insoluble material, the supernatant was analyzed by UV/Vis spectroscopy. CQ in the absence of cells served as negative control.

Smoke exposure. Two p100 mm plates of A549 lung carcinoma cells were aspirated, washed with PBS, and aspirated again. Cigar smoke was blown in 1 plate and rapidly closed so that it was trapped in contact with the cells. After 10 min, cells from both plates were removed with trypsin, pelleted, and a portion re-suspended in 500 μL of the indicated media. 100 mL aliquots were distributed in a white 96-well assay plate and incubated at 37° C. in the CO₂ incubator for 1 h. ATP levels were then analyzed using CellTiterGlo as described previously.

### Example 1

Viruses are not self-sufficient and so must hijack host cells in order to proliferate. This process requires cell entry, detection avoidance, and exploitation of host cellular systems to synthesize/assemble new viral particles. Anti-viral drugs can target unique viral processes or disrupted host systems to prevent virus proliferation, cause host cell death, or illicit an immune response. In some ways viral infection is similar to the cellular transformation underlying cancer, in which genetic changes subvert cellular systems to drive inappropriate proliferation and avoid immune detection. In light of these similarities, transformed h293 kidney epithelial cells were selected as a simple system for analyzing CQ/CHQ effects. The structures of CQ and HCQ are quite similar (FIG. 1A), and most of the following experiments were carried out using CQ. Key results were repeated with HCQ to confirm it was acting in a similar manner. Cells were treated with various concentrations of CQ overnight and evaluated for effects on viability, which often decreases if essential intracellular systems are compromised. Cell viability was measured using CellTiterBlue (CTB), a commonly used approach in which living cells convert resozurin to the fluorescent resorufin via a redox reaction. CQ had minimal effect on cell viability (FIG. 1B, lane 1-6). Because CTB does not lyse cells, it was possible to confirm these results on the exact same cells using CellTiterGlo (CTG), which measures cell viability as a function of ATP concentration. Surprisingly, however, a clear CQ dependent decrease was observed (FIG. 1B, lanes 7-12). These results suggested CQ was targeting cell metabolism. Alternatively, CQ could simply be inhibiting the luciferase in CTG, which uses ATP to produce light. This latter concern was ruled out by showing the same CQ concentration failed to inhibit a luciferase reaction carried out in the absence of cells and containing exogenously added ATP (FIG. 1C).

ATP production in tissue culture cells is an ongoing dynamic process that continuously metabolizes nutrients in the culture media (typically glucose) via glycolysis (which takes place in the cytoplasm) and/or the TCA cycle/Electron Transport Chain (ETC) (which take place in mitochondria). As such it is highly responsive to disruption of these pathways by inhibitors or nutrient availability. To rule out the possibility that overnight CQ exposure was indirectly inhibiting ATP production, the titration was repeated and incubated for only 2 h. No effect on ATP levels was observed (FIG. 2A). Because h293 cells are immortalized and transformed, they operate under conditions of aerobic glycolysis (Warburg effect), in which ATP is generated mainly via glycolysis rather than TCA/ETC. This can be visualized by looking at the change in media color over time (FIG. 2B), which indicates increasing acidity due to production of lactic acid as a glycolysis byproduct. The well-known glucose analog 2-deoxy glucose (2-DG) was therefore employed to ensure that inhibiting glycolysis would rapidly decrease ATP levels. Surprisingly, 2-DG had minimal effect on ATP levels (FIG. 2C, compare lanes 1 and 2). The ETC inhibitor rotenone was therefore evaluated in case cells were undergoing oxidative phosphorylation, but it also had no effect (FIG. 2C, compare lane 1 and 3). It was only when 2-DG and rotenone were combined that ATP decreased dramatically (FIG. 2C, lane 4).

These results indicate that h293 cells can switch between glycolysis and the TCA/ETC, if necessary, in order to maintain ATP levels. Furthermore, since glycolysis is inhibited by 2-DG it cannot be the source of starting material (i.e., pyruvate) for the TCA cycle. Amino acids are the likely alternative, since they are present in the media and can be directed into the TCA cycle after deamination. Support for this idea comes from evaluation of ATP production in media lacking glucose but containing amino acids, which now showed complete inhibition by rotenone (FIG. 2C, compare lanes 5 and 7). Surprisingly, 2-DG also showed significant inhibition (FIG. 2C, lane 6). This could be due to inhibiting metabolism of residual glucose, but great care was taken to avoid possible sources of contamination such as trypsin (used to remove cells) and FBS (Fetal Bovine Serum), both of which typically contain glucose. Cells were also pre-incubated for 30 minutes in the indicated media before adding metabolic inhibitors, which should rapidly deplete any residual glucose. An alternative possibility under investigation is that because 2-DG is phosphorylated and retained in the cytoplasm, its accumulation may indirectly inhibit the TCA/ETC. Regardless, adding glucose back to cells metabolizing amino acids re-established glycolysis and the ability to switch metabolic pathways (FIG. 2C, lanes 9-12).

The effects of CQ on individual metabolic pathways was re-evaluated using 2-DG or rotenone. FIG. 2D shows that titrating CQ had no effect on ATP levels in DMEM media containing both glucose and amino acids (lanes 1-6). Likewise, when cells were forced to use the glycolytic pathway by including rotenone to inhibit the ETC, CQ again had no effect (FIG. 2D, lanes 13-18). However, ATP levels were markedly reduced by CQ in the presence of 2-DG (FIG. 2D, lanes 5-8). Because 2-DG inhibits glycolysis and forces cells to produce ATP via the TCA/ETC, these results suggest CQ is specifically targeting oxidative phosphorylation. Similar data was obtained with HCQ, indicating it is working in a similar manner (FIG. 2E). These results were also confirmed in immortalized but untransformed human diploid fibroblasts (FIG. 2F), indicating that CQ effects in the presence of 2-DG are not confined to h293 cells. As a complementary approach CQ activity was evaluated in DMEM containing amino acids but lacking glucose, which as shown previously forces cells to use the TCA/ETC (FIG. 2C). CQ alone now inhibited ATP production (FIG. 3A, lanes 1-6), consistent with inhibition of oxidative phosphorylation. Repeating the experiment in the presence of glucose showed minimal effect on ATP levels (FIG. 3A, lanes 7-12), which could be due to preventing CQ inhibition of the TCA/ETC or by providing an alternative pathway for ATP production. To distinguish between these possibilities, an order of addition experiment was performed to determine if activation of glycolysis could bypass CQ effects. Cells were first incubated with CQ in media lacking glucose to allow inhibition of ATP production (FIG. 3B, lanes 1-6). Glucose was then added for 1 h, which re-established ATP levels via glycolysis (FIG. 3B, lanes 7-12). Thus, activation of glycolysis can bypass CQ inhibition of the TCA/ETC. Conversely, glycolysis does not prevent CQ inhibition of oxidative phosphorylation. When cells were incubated in L-15 media containing glucose and treated with CQ, the addition of 2-DG re-established inhibition of ATP production (FIG. 3B, lanes 13-18). Given that the TCA/ETC is located in mitochondria, and the known ability of CQ to become trapped in acidic compartments, it seemed possible CQ was inhibiting ATP production by sequestering protons in the mitochondrial intermembrane space.

An alternative explanation, however, is that CQ inhibits amino acid metabolism, which is responsible for ATP production in the presence of 2-DG (or absence of glucose). To address this possibility cells were cultured in media substituting galactose for glucose (L-15 media). Galactose is converted to glucose-6-P and metabolized through the glycolytic pathway, but because this conversion costs 2 ATP, the resulting pyruvate must go through the TCA/ETC in order to generate net ATP. FIG. 3C shows that ATP production in L-15 media (containing galactose instead of glucose) is partially inhibited by 2-DG and completely inhibited by rotenone, as expected (lanes 1-4). Supplementing L-15 with glucose re-establishes the metabolic switch as indicated by synergy between 2-DG and rotenone (FIG. 3C, lanes 5-8). CQ and HCQ were then titrated on cells in L-15 media in the absence and presence of glucose (FIG. 3D). Both CQ (FIG. 3D, lanes 1-6) and HCQ (FIG. 3D, lanes 13-18) inhibited ATP production when cells were dependent on oxidative phosphorylation. In marked contrast, adding glucose to the L-15 media prevented inhibition by allowing cells to utilize the glycolytic pathway (FIG. 3D, lanes 7-12 and 19-24). Taken together these observations indicate CQ and HCQ are not simply targeting amino acid metabolism, but rather inhibiting the TCA/ETC in mitochondria.

To determine whether mitochondrial inhibition was due to the ability of CQ to become trapped in acidic compartments, the pH dependency of CQ activity was investigated in more detail. An important clue in this regard was initial experiments using the L-15 galactose media described earlier. When this media was used as purchased and incubated in a 37° C. CO₂ incubator, no CQ inhibition was observed (FIG. 4A). Note that cells were still sensitive to 2-DG and rotenone, as expected (FIG. 4A, lanes 7 and 8). Visual inspection revealed, however, that the media was rapidly acidified in the CO₂ incubator because it lacks sodium bicarbonate (FIG. 4B). When L-15 media was properly buffered with sodium bicarbonate, CQ inhibition in the CO₂ incubator was re-established (FIG. 3D). These results suggested that CQ activity is strongly pH dependent and were consistent with previous work showing the free base form (FIG. 4C) is preferentially internalized in cellular compartments. To test this idea directly, a DMEM media lacking sodium bicarbonate was generated from individual ingredients. Cells in the absence and presence of CQ were then incubated in this media adjusted to pH 7 to 10 using 25 mM Hepes for 1 h. Cells were then washed, re-fed with L-15 media (containing galactose), and incubated at 37° C. plus CO₂ to allow ATP production via oxidative phosphorylation. As shown in FIG. 4D, there was a dramatic increase in CQ inhibition with increasing pH, consistent with the unprotonated form entering cells and accumulating in mitochondria. Since CQ exists in equilibrium between its various forms (FIG. 4C), inhibition might be time dependent. Cells were therefore incubated in L-15 media (plus sodium bicarbonate) without and with CQ for various times, followed by analysis of ATP levels. FIG. 4E shows that the amount of CQ inhibition increased dramatically over time, consistent with its slow entry into cells and accumulation in the mitochondria.

To more directly confirm that CQ was entering cells, h293s were incubated in L-15 media with a fixed CQ concentration for 2 h in the absence and presence of glucose to determine if glycolysis affected CQ location. Cells alone and CQ alone were analyzed as controls. After pre-incubation, cells were pelleted and washed extensively to remove any unincorporated CQ, then re-suspended in buffer and separated into nuclei, cytoplasm, and mitochondrial fractions by dounce homogenization. Extracts from the various fractions were evaluated by UV/Vis spectroscopy and the resulting profiles compared to that of pure CQ (FIG. 4F). Panel 1 shows the spectrum for pure CQ. The two distinctive peaks at ˜330 and 345 nm are outside the range of major biological components (e.g., nucleic acids and proteins) and hence can serve as indicators of CQ presence. Panel 2 shows the spectra for the nuclear fraction, where there is no evidence of CQ. In marked contrast, UV/Vis analysis of the cytoplasmic fraction clearly showed the two peaks indicating the presence of CQ (panel 3). Interestingly, cells incubated in L-15 plus glucose contained significantly more CQ than cells alone. This likely reflects the fact that h293s undergo aerobic glycolysis (see FIG. 2B), which requires converting pyruvate to lactic acid in order to maintain ATP synthesis. This reaction may transiently lower the cytoplasmic pH, resulting in CQ protonation and retention in the cytoplasm. Panel 4 shows analysis of the mitochondrial fraction, whose volume is much smaller than that of the nuclear and cytoplasmic components (note the difference in Absorbance values). Nevertheless, cells incubated with CQ show a modest accumulation in the mitochondrial fraction, while including glucose results in a dramatic increase in the CQ signature. While this result might seem counterintuitive given panel 3, increasing CQ in the cytoplasm increases the probability that it will eventually become trapped in the more acidic mitochondrial intermembrane space.

Red blood cells (RBCs), which lack both mitochondria and nuclei, were utilized to determine more directly whether: 1) CQ can be trapped in the cytoplasm, and 2) this inhibits glycolysis. RBCs were re-suspended in DMEM containing 5 mM glucose (physiological concentration) and incubated in the absence or presence of CQ. Cells were then pelleted by centrifugation and washed with the same DMEM incubation media lacking CQ. At this point a small fraction of cells was withdrawn for analysis of ATP levels. Remaining cells were pelleted again and re-suspended in water for lysis. Extracts were then evaluated by UV/Vis to determine if CQ was present. FIG. 4G shows the spectra for pure CQ, RBCs without CQ, and RBCs plus CQ. Cells pre-incubated with CQ clearly contain the two peaks at 330 and 345 nm, indicating CQ was retained in the cytoplasm. Since RBCs generate ATP solely by glycolysis, this result is consistent with the h293 fractionation studies demonstrating increased cytoplasmic CQ when glucose was present (FIG. 4F, panel 3). The inset graph shows that the presence of CQ in RBCs had no effect on ATP production via glycolysis, consistent with the h293 data indicating CQ preferentially targets metabolic processes in the mitochondria (e.g., FIG. 3B). It should be emphasized that this and most other experiments with h293s were demonstrating that short term exposure to CQ does not inhibit glycolysis. However, higher concentrations and longer incubation times may allow CQ to build up sufficiently that glycolysis is also affected (see for example FIG. 1B).

Accumulated data suggest that CQ/HCQ cell entry and accumulation in the mitochondria is determined by its protonation status. If this view is correct, then structurally distinct compounds with similar protonatable nitrogens might operate in similar fashion. The first molecule examined was quinine, the original malarial drug from which CQ was derived. It also has two nitrogens available for protonation, but is overall structurally distinct (FIG. 5A). Quinine was titrated against h293s plated in DMEM alone or with 2-DG and rotenone, whereupon ATP levels were evaluated. FIG. 5B shows that quinine inhibition was much more dramatic in the presence of 2-DG, just as was observed with CQ (FIG. 2D). These results suggest that quinine functions like CQ, accumulating in the mitochondria in a pH-dependent manner. Encouraged by these results, other potential modulators of mitochondrial function were evaluated. Nicotine (FIG. 5C) was of particular interest because: 1) epidemiological studies indicate that smokers may actually be protected from SARS-CoV-2 infection; and 2) it contains protonatable nitrogens. FIG. 5D shows that despite having to use high concentrations of purified nicotine, there does appear to be specificity for inhibiting mitochondrial ATP production. Attempts to enhance this effect by increasing the media pH (to generate the free base) or by using different cells lines (which might be more amendable to nicotine uptake), gave results similar to those in h293s. As a last resort, cigar smoke containing nicotine was blown directly on A549 lung carcinoma cells, followed by analysis of ATP production in different media. Remarkably, ATP levels were only reduced when cells were forced to use the TCA/ETC (L-15 media). This does not appear to be a viability effect because cells in DMEM and L-15 plus glucose were unaffected. These results suggest either that vaporization is essential for generating the version of nicotine able to accumulate in mitochondria, or perhaps more intriguingly, that some other component in smoke is responsible.

Understanding a drug's biochemical mechanism of action in relation to its structure allows one to identify other molecules that might behave in a similar fashion. A molecule of interest in this regard was diphenhydramine, the active ingredient in Benadryl. Although it has not been implicated as a possible COVID-19 therapeutic, it contains a nitrogen functional group that is structurally quite similar to that of CQ (FIG. 5C). As shown in FIG. 5D, diphenhydramine had minimal effect on ATP production under conditions where cells can switch between glycolysis and TCA/ETC, but strongly inhibited when TCA/ETC was the only option (note the much lower concentrations utilized compared to nicotine). In marked contrast, famotidine (FIG. 5E), another commercial drug implicated as a possible COVID-19 therapeutic, had no effect on ATP production in the absence or presence of 2-DG despite its many nitrogens (FIG. 5F). It therefore seems likely that the relatively greater hydrophobicity associated with molecules targeting the mitochondria is an important structural element required for their activity. These results therefore support a model in which CQ and related compounds are distinguished by the following functionalities: 1) constituents that provide an ability to pass through membranes, and 2) the presence of nitrogens with pKa's that allow formation of the free base uncharged form, which after passing thru the membranes can become protonated and trapped in acidic environments, such as the mitochondrial intermembrane space. At sufficiently high concentrations of CQ/HCQ, these events lead to depletion of protons needed for ATP synthesis.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of treating a patient having a coronavirus infection or preventing a coronavirus infection in a patient, the method comprising administering to the patient an effective amount of a first pharmaceutical therapy comprising a glycolysis inhibitor and a second pharmaceutical therapy comprising a compound having one or more aromatic rings and at least one alkylamino_{(C≤12)} group or substituted alkylamino_{(C≤12)} group.

2. The method of claim 1, wherein the glycolysis inhibitor is 2-deoxy-D-glucose (2-DG).

3. The method of claim 1, wherein the compound is epinephrine, diphenhydramine, diphenhydramine hydrochloride, chloroquine, hydroxychloroquine, 3,4-methylenedioxymethamphetamine, chlorpromazine, phenylephrine, disulfiram, rosiglitazone, camostat, clemastine, dextromethorphan, or nicotine.

4. The method of claim 1, wherein the compound is in its free base form.

5. The method of claim 1, wherein the first pharmaceutical therapy is administered systemically.

6. The method of claim 1, wherein the first pharmaceutical therapy is administered orally, by inhalation, by injection, intraarterially, or intravenously.

7. The method of claim 1, wherein the first pharmaceutical therapy is administered at a dosage of from about 25 mg/kg and to about 150 mg/kg.

8. The method of claim 1, wherein the first pharmaceutical therapy is administered daily.

9. The method of claim 1, wherein the second pharmaceutical therapy is administered systemically.

10. The method of claim 1, wherein the second pharmaceutical therapy is administered orally, by inhalation, by injection, intraarterially, or intravenously.

11. The method of claim 1, wherein the first pharmaceutical therapy is administered prior to the administration of the second pharmaceutical therapy.

12. The method of claim 1, wherein the first pharmaceutical therapy and the second pharmaceutical therapy are administered concurrently.

13. The method of claim 1, wherein the first pharmaceutical therapy and the second pharmaceutical therapy are administered by distinct routes.

14. The method of claim 1, wherein the coronavirus is a betacoronavirus.

15. The method of claim 1, wherein the coronavirus is a severe acute respiratory syndrome-related (SARS) coronavirus.

16. The method of claim 1, wherein the coronavirus is SARS-CoV-2.

17. The method of claim 16, wherein the method is a method of treating or preventing COVID-19 in a patient in need thereof.

18. The method of claim 16, wherein the method inhibits replication of SARS-CoV-2 in the patient.

19. The method of claim 1, further comprising administering to patient a second therapy.

20. The method of claim 19, wherein the second therapy comprises administering to said patient a therapeutically effective amount of a second drug.

21. The method of claim 20, wherein the second drug is an anti-platelet drug, an anti-coagulation agent, an anti-viral drug, a corticosteroid, a human type I IFN, or a human type III IFN.

22. The method of claim 20, wherein the second drug is remdesivir.

23. A pharmaceutical composition comprising:
(a) a glycolysis inhibitor;
(b) a compound having one or more aromatic rings and at least one alkylamino_{(C≤12)} group or substituted alkylamino_{(C≤12)} group; and
(c) a pharmaceutically acceptable carrier.

24. The composition of claim 23, wherein the glycolysis inhibitor is 2-deoxy-D-glucose (2-DG).

25. The composition of claim 23, wherein the compound having one or more aromatic rings and at least one alkylamino_{(C≤12)} group or substituted alkylamino_{(C≤12)} group is epinephrine, diphenhydramine, diphenhydramine hydrochloride, chloroquine, hydroxychloroquine, 3,4-methylenedioxymethamphetamine, chlorpromazine, phenylephrine, disulfiram, rosiglitazone, camostat, clemastine, dextromethorphan, or nicotine.

26. The composition of claim 23, wherein the compound having one or more aromatic rings and at least one alkylamino_{(C≤12)} group or substituted alkylamino_{(C≤12)} is in its free base form.

27. A kit of part, the kit comprising:
(a) a first pharmaceutical composition comprising a glycolysis inhibitor;
(b) a second pharmaceutical composition comprising a compound having one or more aromatic rings and at least one alkylamino_{(C≤12)} group or substituted alkylamino_{(C≤12)} group,
wherein the first pharmaceutical composition and the second pharmaceutical composition are provided as separate components of the kit.

28. The kit of claim 27, wherein the glycolysis inhibitor is 2-deoxy-D-glucose (2-DG).

29. The kit of claim 27, wherein the compound having one or more aromatic rings and at least one alkylamino_{(C≤12)} group or substituted alkylamino_{(C≤12)} group is epinephrine, diphenhydramine, diphenhydramine hydrochloride, chloroquine, hydroxychloroquine, 3,4-methylenedioxymethamphetamine, chlorpromazine, phenylephrine, disulfiram, rosiglitazone, camostat, clemastine, dextromethorphan, or nicotine.

30. The kit of claim 27, wherein the compound having one or more aromatic rings and at least one alkylamino_{(C≤12)} group or substituted alkylamino_{(C≤12)} is in its free base form.
